(19) United States
(12) Patent Application Publication
(10) Pub. No.: US 2024/0197803 A1
(43) Pub. Date: Jun. 20, 2024

(54) USE OF MANGOSTEEN FRUIT SHELL EXTRACT IN THE PREPARATION OF A MEDICAMENT FOR TREATING PSORIASIS

(71) Applicant: Xantho Biotechnology Co., LTD, Taipei City (TW)

(72) Inventors: Huan-Yuan CHEN, Taipei City (TW); Shih-Yin CHEN, New Taipei City (TW); I-Pin CHUANG, New Taipei City (TW); Ku-Cheng CHEN, New Taipei City (TW); Yen-Ju CHEN, New Taipei City (TW)

(73) Assignee: Xantho Biotechnology Co., LTD, Taipei City, TW (TW)

(21) Appl. No.: 18/566,672

(22) PCT Filed: Nov. 5, 2021
(86) PCT No.: PCT/CN2021/128869

§ 371 Date: Dec. 4, 2023

(51) Int. Cl. A61K 36/38; A61K 31/352; A61P 17/06

(52) U.S. Cl. CPC A61K 36/38; A61K 31/352; A61P 17/06; A61K 2236/331; A61K 2236/333

(57) ABSTRACT

A method for treating psoriasis is provided, including administering a pharmaceutical composition to a subject suffering from psoriasis, wherein the pharmaceutical composition comprises an effective amount of mangosteen fruit shell extract, the mangosteen fruit shell is outer shell of the mangosteen fruit shell and/or inner shell of the mangosteen fruit shell, and the pharmaceutical composition is an external preparation.

### CROSS-REFERENCE TO RELATED APPLICATION

This application is a national stage application, filed under 35 U.S.C. § 371, of International Patent Application No. PCT/CN2021/128869, filed on Nov. 5, 2021, which is incorporated by reference herein in its entirety.

### FIELD OF THE INVENTION

The present invention relates to a use of Mangosteen fruit shell extract in the preparation of a medicament for treating psoriasis.

### BACKGROUND OF THE INVENTION

Skin is the largest organ of the human body. There are many types of skin diseases. Skin diseases may be acute (lasting only a few minutes to several hours) or chronic conditions, which may affect individuals for days, months, years and even the entire life. Skin diseases may be conditions caused by fungal, bacterial, or viral sources, or may be non-infectious, immune responses, such as inflammatory reactions with or without allergens, or idiopathic. Therefore, the symptoms of the skin diseases may vary and range from mild itching, redness and swelling to severe pus and nociceptive pain, for examples damaging ulceration. Skin diseases may impose significant impact on the quality of an individual's life.

Psoriasis is a common chronic inflammatory skin-related disease, affecting around 3% of world population. The onset of psoriasis could be resulted from multiple factors, including genetic, epigenetic, environmental, and lifestyle factors, among them involving both innate and adaptive immune responses. Once the immunity processes are activated, more dendritic cells, macrophages, and T cells are recruited from the lesion skins and secrete more inflammatory mediators. This process in turn precipitates the epidermal hyper-proliferation and the aberrant differentiation of keratinocytes. As a consequence, the epidermis is thickened, and the psoriatic plaques are caused.

The treatment of psoriasis aims to stop skin cells from growing so quickly and to remove scales. There are three therapies currently used, topical therapy (creams and ointments), phototherapy (light therapy) and oral or injected medication. Steroid drugs are commonly used for topical therapy and oral or injected medication, especially in the treatment of moderate to severe psoriasis. However, long-term use or overuse of strong corticosteroids may cause some unpleasant side effects.

Mangosteen has been used in the field of breast cancer prevention and muscle-related diseases, it has also been developed as nutritional supplements and cosmetics in daily lives, as well as uses in the treatment of acute hepatitis, liver fibrosis and cirrhosis prevention.

Matsumoto et al., have studied α-mangostin, β-mangostin, γ-mangostin, and methyl-β-mangostin purified from Mangosteen fruit shells and investigated the inhibitory effect of this compound at various stages of the cell cycle, showing that this compound has anti-cell proliferative effect and anti-tumor effect (Bioorg. Med. Chem. 2005, 13, 6064-6069).

### SUMMARY OF THE INVENTION

The present invention provides a method for treating psoriasis in a subject, comprising administering a pharmaceutical composition comprises an effective amount of mangosteen fruit shell extract.

In a preferred embodiment, the Mangosteen fruit shell is water extract of Mangosteen fruit shell and/or alcohol extract of Mangosteen fruit shell.

In another preferred embodiment, the Mangosteen fruit shell is the outer shell of the Mangosteen fruit shell.

### DETAILED DESCRIPTION OF THE INVENTION

The present invention provides a use of a composition in preparation of a pharmaceutical composition for treating skin disorders.

Specifically, the present invention provides a use of a composition in preparation of a medicament for treating psoriasis, wherein the composition comprises an effective amount of extract of Mangosteen fruit shell. The medicament can also be used for topical treatment use or for precision treatment use.

The present invention provides a method for treating psoriasis in a subject, comprising administering a pharmaceutical composition comprises an effective amount of mangosteen fruit shell extract.

In the present invention, the said psoriasis including plaque psoriasis, nail psoriasis, guttate psoriasis, inverse psoriasis, pustular psoriasis, erythrodermic psoriasis and psoriatic arthritis.

Mangosteen fruit shell contains a softer inner shell and a harder outer shell.

In a preferred embodiment, the Mangosteen fruit shell is extracted with a solvent which is selected from the group consisting of methanol, ethanol, n-propanol, 2-propanol, n-butanol, acetone, ethyl acetate and water.

In another preferred embodiment, the extract of Mangosteen fruit shell is water extract of Mangosteen fruit shell and/or alcohol extract of Mangosteen fruit shell.

In a preferred embodiment, the extract of Mangosteen fruit shell is a Mangosteen fruit shell water extract.

In another preferred embodiment, the extract of Mangosteen fruit shell is Mangosteen fruit shell alcohol extract.

In a preferred embodiment, the Mangosteen fruit shell is the inner shell/outer shell of the Mangosteen fruit shell and/or the whole shell of the Mangosteen fruit shell.

In another preferred embodiment, the Mangosteen fruit shell is the outer shell of the Mangosteen fruit shell.

In a preferred embodiment, the compositions of the present invention can be oral or parenteral preparations, the parenteral preparations can be external preparations which can be creams, pastes, ointments, gels, wash lotions or patches.

In a preferred embodiment, the extract of Mangosteen fruit shell of the present invention comprises α-mangostin and γ-mangostin.

In another preferred embodiment, the water extract of Mangosteen fruit shell of the present invention comprises α-mangostin and γ-mangostin.

In yet another preferred embodiment, the alcohol extract of Mangosteen fruit shell of the present invention comprises α-mangostin and γ-mangostin.

As used herein, the term “Effective amount” is the amount that can achieve effective results when administered to an individual, or that has the desired activity in vivo or in vitro. In the case of psoriasis, as compared to no treatment, effective clinical outcomes include amelioration of the extent or severity of the symptoms associated with the disease or condition, and/or prolonging the life of an individual and/or improvement of the quality of life of the individual. The exact amount of compound administered to an individual will depend on the type and severity of the disease or symptoms and on the individual characteristics such as the general health of the individual, age, sex, weight, and drug tolerance of the individual. It is also dictated by the conditions, severity and types of the inflammatory disorder, the autoimmune disorder and the allergic disorder, or the desired immunosuppressive effect. Those skilled in the art will be able to determine the appropriate dose based on these and other factors.

In a preferred embodiment, the effective amount of extract of Mangosteen fruit shell is 1% (w/w) to 10% (w/w). In a most preferred embodiment, the effective amount of extract of Mangosteen fruit shell is 1.25% (w/w) to 5% (w/w).

The pharmaceutical composition of the present invention can be formulated into various forms of oral or parenteral preparations. Oral preparations can be formulated as solid preparations such as powders, granules, troches, capsules, etc., or formulated as liquid preparations such as suspensions, emulsions, syrups, etc. Parenteral preparations can be formulated as external preparations such as creams, ointments, gels, wash lotions, patches, etc., or as inhalants, aerosols, suppositories, etc.

The pharmaceutical composition of the present invention can comprise pharmaceutically acceptable excipients, especially can further comprise predetermined solvents or oils, PH adjuster and if desired, can further comprise a dispersant.

Examples of solvents used in the present invention include, but are not limited to, water, ethanol, isopropanol, 1,3-butanediol, propylene glycol, glycerin, etc.

Examples of oils used in the present invention are selected from the group consisting of, but are not limited to, corn oil, sesame oil, flaxseed oil, cottonseed oil, soybean oil, peanut oil, mono-glycerides, di-glycerides, tri-glycerides, mineral oil, squalene, jojoba oil, olive oil, evening primrose oil, borage oil, grape seed oil, coconut oil, sunflower oil, shea butter, and any combinations thereof.

Solvents and oils can be used alone or in any combinations thereof.

Examples of useful dispersants include, but are not limited to, lecithin, organic monoglycerides, sorbitan fatty acid esters, polyoxyethylene fatty acid esters, sorbitan stearate, etc. These raw materials can also be used alone or in any combinations thereof.

If desired, the composition further comprises additional materials such as antimicrobials or preservatives.

In the meantime, it is known that an active ingredient can be used simultaneously with the composition as long as it does not have any adverse effects on the pharmaceutical activity of the composition of the present invention. For example, ceramide moisturizers are commonly used as conventional agents for atopic dermatitis, or liquid ingredients such as hydrocortisone steroids, vitamin A derivatives such as vitamin A palmitate and/or tocopherol, etc., can be used with the composition.

When the pharmaceutical composition is used as an external preparation, an appropriate external skin preparation can be used as a base material, and an aqueous solution, a non-aqueous solvent, a suspension, an emulsion or a lyophilized preparation, etc., can be used and sterilized according to known methods.

In practical use of the provided or administered composition of the present invention, the dosage can be determined depending on various factors such as the route of administration, the age, sex, and weight of the patient, the severity of the disease, and the type of medicament as the active ingredient.

In the case where the composition of the present invention can be a food or a cosmetic composition, the composition can be prepared by appropriate addition of at least one food supplement or a cosmetically acceptable carrier.

The food composition can be used in or added to, for example, healthy foods. As used herein, the term “healthy food” refers to a food product containing the composition of the present invention that has an enhanced function as compared to general food products. Healthy foods can be prepared by adding a general food to the composition or by encapsulation, pulverization or suspension liquefaction.

The cosmetic composition can be added alone or together with other cosmetic ingredients, or can be appropriately used according to other known methods. Cosmetics include, but are not limited to, aftershaves, lotions, creams, facial masks and color makeups.

Cosmetic compositions can be formulated into various forms of compositions, such as gels, creams, ointments, etc. The compositions in the form of gels, creams and ointments can be appropriately prepared according to the form of the composition by using known methods, and by addition of known softeners, emulsifiers and thickeners or other materials known in the art.

The gel-form composition can be prepared, for example, by addition of a softener such as trimethylolpropane, polyethylene glycol and glycerol, for example, a solvent of propylene glycol, ethanol and isocetyl alcohol, and pure water.

The preparation of the compositions in the form of creams can be carried out, for example, by addition of fatty alcohols such as stearyl alcohol, myristyl alcohol, behenyl alcohol, resveratrol, isostearyl alcohol and isocetyl alcohol; emulsifiers such lipids, as as such lecithin, phosphatidylcholine, phosphatidylethanolamine, phosphatidyl serine, phosphoinositide and derivatives thereof, glyceryl stearate, sorbitol palmitate, sorbitol stearate, etc; natural fats And oils such as avocado oil, almond oil, babassu oil, borage oil, camellia oil, etc; lipid compositions such as ceramides, cholesterol, fatty acids, phytosphingosine, lecithin, etc; solvents, such as propylene glycol, etc; and pure water.

The preparation of the compositions in the form of ointments can be carried out, for example, by addition of emollients, emulsifiers and waxes, for example microcrystalline wax, paraffin, ceresin, beeswax, spermaceti, petrolatum, etc.

In another aspect, the present invention provides a method for using the composition to prepare a medicament for treating or alleviating psoriasis. As used herein, the term “treating or alleviating” means that when a patient uses a medicament, it stops or delays the course or symptoms of the disease.

### BRIEF DESCRIPTION OF THE DRAWINGS

FIG. 1 shows the comparison of ear thickness change of IL-23 injected right ear with placebo compared with PBS injected left ear.

FIG. 2A-FIG. 2D illustrate the curative effect of 3 ointment samples to psoriasis. FIG. 2A indicates that all the ointment samples did not cause any adverse effect; FIG. 2B, FIG. 2C and FIG. 2D show 3 ointment samples had the potential to treat psoriasis. Abbreviation: W: whole shell extracts.

FIG. 3A-FIG. 3B illustrate that all the ointment samples significantly reduce epidermal thickening. FIG. 3A shows the histology of ear tissue sections of PBS/IL-23 injected mice using H&E staining. Scale bar, 50 μM. FIG. 3B shows the ear thickness of upper (dorsal) layer are measured. Results are expressed as the mean±SEM. *P<0.05, **P<0.01, ***P<0.001, Student's t test. Abbreviation: W: whole shell extracts.

FIG. 4A-FIG. 4C illustrate the curative effects of 5% inner shell extracts and 5% outer shell extracts. FIG. 4A shows both 5% inner shell extracts and 5% outer shell extracts show the potential to treat psoriasis, and the curative effect of 5% outer shell extracts was better than the curative effect of 5% inner shell extracts. FIG. 4B and FIG. 4C show 5% inner shell extracts and 5% outer shell extracts significantly reduce epidermal thickening. Results are expressed as the mean±SEM. *P<0.05, ***P<0.001, Student's t test. Abbreviation: W: whole shell extracts; I: inner shell extracts; O: outer shell extracts.

### EMBODIMENTS

The examples below are non-limiting and are merely representative of various aspects and features of the present invention.

### Examples

### Preparation of Pharmaceutical Compositions

Mangosteen fruit shell was collected and dried to 50% to 95%, extracted with a solvent (such as water or 10% to 95% alcohol), and concentrated to obtain an extract of Mangosteen fruit shell.

The outer shell and inner shell of the Mangosteen fruit shell were separated, the outer shell of the Mangosteen fruit shell and the inner shell of the Mangosteen fruit shell were respectively dried to 50% to 95%, and extracted with a solvent (such as water or 10% to 95% alcohol), then concentrated to obtain an extract of mangosteen outer shell and an extract of mangosteen inner shell.

Different concentrations of pastes or ointments were prepared from the Mangosteen fruit shell alcohol and water extract, the mangosteen inner shell, outer shell alcohol and water extract.

### Establishment of IL-23-Induced Psoriasis Mouse Model

Male Wild-Type mice (C57BL/6), age 8-11 weeks, were used for study. Mice were anesthetized by 1%-3% isoflurane with 100% oxygen. To induce the psoriasis-like pathogenesis, the right ear of mice was be injected with IL-23 and the left ear was be injected with control buffer (PBS) as previously described. Intradermal inject carrier-free recombinant mouse IL-23 (1 μg in 10 μl; eBioscience, cat. No. 34-8231-85) into the right ear by using a 31-guagae needle. The injection will be continued every other day for 14 days to induce the psoriasis-like disorder. Sterile PBS was be injected into the left ear of mice as vehicle control.

### Animal Experiment

60 mice were used for animal experiment, including 48 psoriasis-like disorder induced mice and 12 non-induced mice. All the mice were separated to two tests, 30 for each test. Each test including 5 groups, group 1 (6 psoriasis-like disorder induced mice treated with 1.25% Whole shell extracts), group 2 (6 psoriasis-like disorder induced mice treated with 2.5% Whole shell extracts), group 3 (6 psoriasis-like disorder induced mice treated with 5% Whole shell extracts), group 4 (6 psoriasis-like disorder induced mice treated with placebo), and group 5 (6 non-induced mice administered with PBS only). The two tests and the group assignments are shown in Table 1.

TABLE 1

| | | | | |
|---|---|---|---|---|
| | | | | Sample |
| Group | Injection | Number of | | Dose |
| No. | (1 μg in 10 μL) | Animals (N) | Ointment Sample | (mg/cm²) |

Test 1

| | | | | |
|---|---|---|---|---|
| 1 | IL-23 | 6 | 1.25% Whole shell | 40.25 |
| | | | extracts | |
| 2 | IL-23 | 6 | 2.5% Whole shell | 40.25 |
| | | | extracts | |
| 3 | IL-23 | 6 | 5% Whole shell | 40.25 |
| | | | extracts | |
| 4 | IL-23 | 6 | placebo | 40.25 |
| 5 | PBS | 6 | None | NA |

Test 2

| | | | | |
|---|---|---|---|---|
| 1 | IL-23 | 6 | 1.25% Whole shell | 40.25 |
| | | | extracts | |
| 2 | IL-23 | 6 | 2.5% Whole shell | 40.25 |
| | | | extracts | |
| 3 | IL-23 | 6 | 5% Whole shell | 40.25 |
| | | | extracts | |
| 4 | IL-23 | 6 | placebo | 40.25 |
| 5 | PBS | 6 | None | NA |

Sterile PBS was be injected into the left ear of group 5 mice as vehicle control. Placebo (group 4) and 3 ointment samples (group 1-group 3) were applied on the injection point (40.25 mg/cm2 each ear) every day from day 0 to day 13. All the mice were sacrificed on day 14. Ear thickness was measured at the center of ears through a pocket thickness gage (Mitutoyo Corp.) every other day. Ear photos were taken on day 0, day 5, day 9 and day 14 (data not shown).

### Hematoxylin and Eosin (H&E) Staining and Epidermal Thickness Measurement

At the end of the experiments, mice were sacrificed and the injected ears were harvested. Half of the ears were fixed in 10% formalin and embedded in paraffin, sectioned, and stained with hematoxylin and eosin. The mean epidermal thickness was determined on H&E-stained sections by measuring the distance between the outermost surface of the epidermis excluding the stratum corneum and the dermal-epidermal junction at five randomly chosen sites in each sample through Olympus cellSens Standard software.

### Result

The effects of 3 ointment samples with IL-23 induced psoriasis mouse model were tested. Right ears of mice were injected with lug carrier-free recombinant mouse IL-23 every other day for 14 days, and left ears were injected sterile PBS vehicle control.

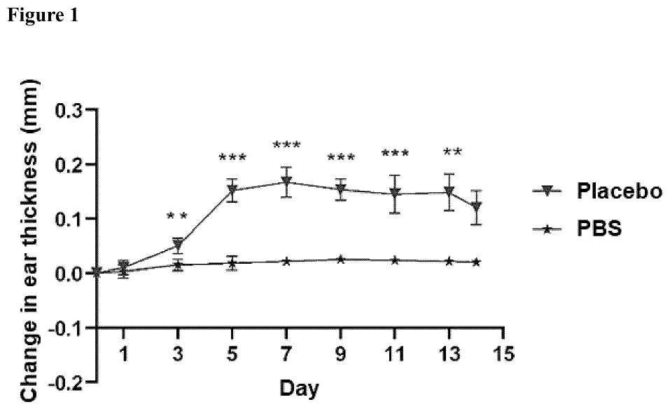

Placebo and 3 ointment samples (40.25 mg/cm² each ear) were applied on the injection point every day. Ear photos were taken on day 0, day 5, day 9 and day 14. To check the effects of ointment samples on ear swelling, we measured ear thickness every other day. The ear thickness of IL-23 injected right ear with placebo showed significant change compared with the ear thickness of PBS injected left ear from day 3 to day 13 (FIG. 1). The result indicated that we used IL-23 injection could successfully induced mouse ear swelling and this was one of symptoms in psoriasis.

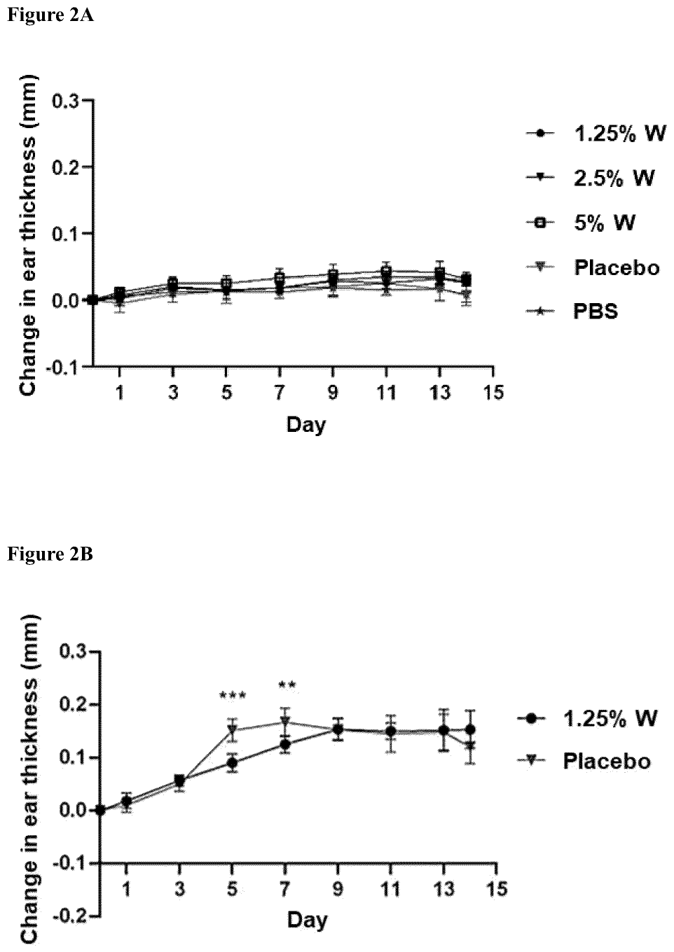

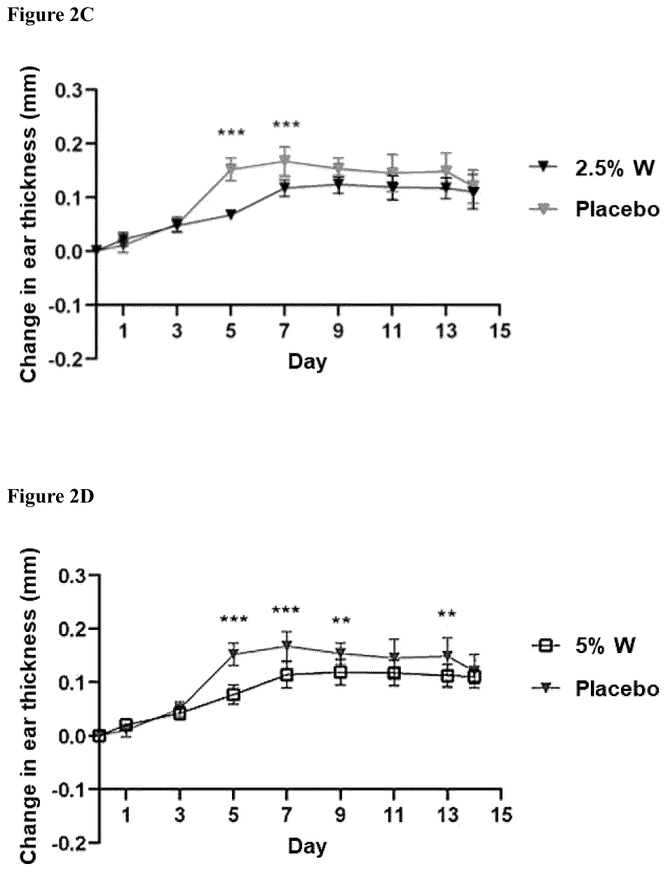

The ear thickness of PBS injected left ear in all groups did not show significant change during day 0 day 14 (FIG. 2A), which indicated that all the ointment samples did not cause any adverse effect.

The ear thickness of IL-23 injected right ear with 3 doses of ointment samples were shown in FIGS. 2B-2D. All of them significantly reduced ear swelling on day 5. One of them, 5% whole shell extracts, reduced ear swelling for a long-time during day 5 to day 9 and on day 13.

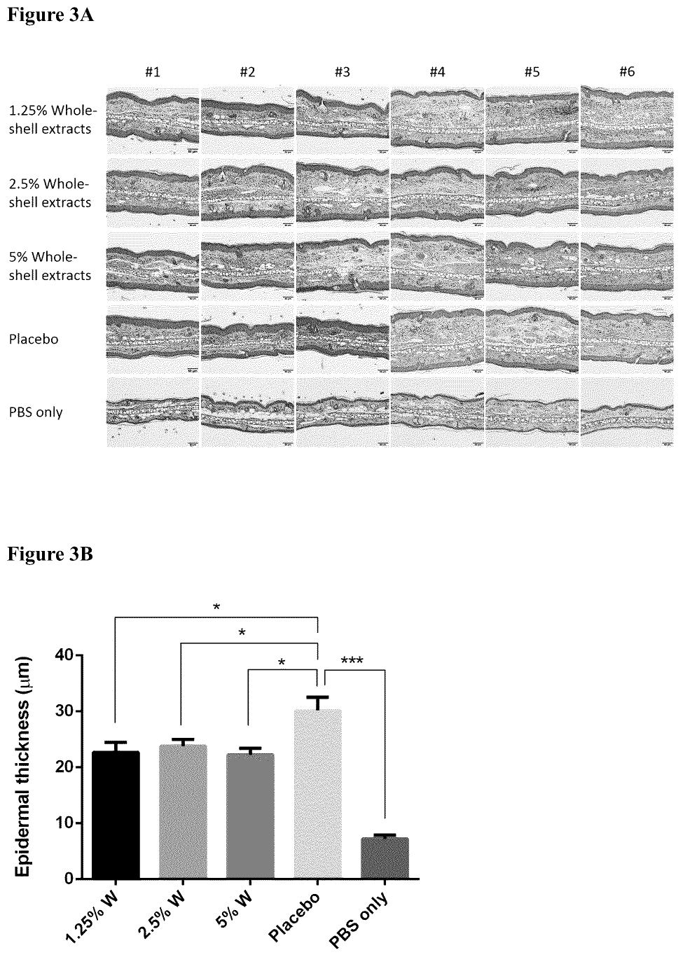

Histological analyses were performed on ear sections obtained from sacrificed mice. H&E staining revealed the characteristic of the psoriasis-like phenotype in placebo group, such as thickened epidermis, epidermal rete-ridge projections into the dermis and many cells infiltration to the inflammation site (FIG. 3A). The groups that applied ointment samples educed epidermis thickness and cells infiltration were slightly reduced.

The epidermal thickness of each mouse was measured (FIG. 3B). These results also showed that the thickness was significantly increase in placebo group compare with PBS only group. In contrast to the result of placebo group, all ointment samples, including 1.25% Whole shell extracts, 2.5% Whole shell extracts, 5% Whole shell extracts, significantly reduced epidermal thickness. The H&E staining results showed that in psoriasis-like mouse model ointment samples could significantly reduce epidermal thickness and slightly reduce the psoriasis-like phenotype in histological analyses.

It was worth noting that all the ointment sample applied groups showed early curative effect in 5 days, especially, administering with 5% Whole shell extracts also showed the reduction of the ear thickness for lasting longer time.

To further evaluated which part showed mainly effective for treating psoriasis, 5% outer shell of the mangosteen shell and 5% inner shell of the mangosteen shell were used for the same experiment and compared with all groups.

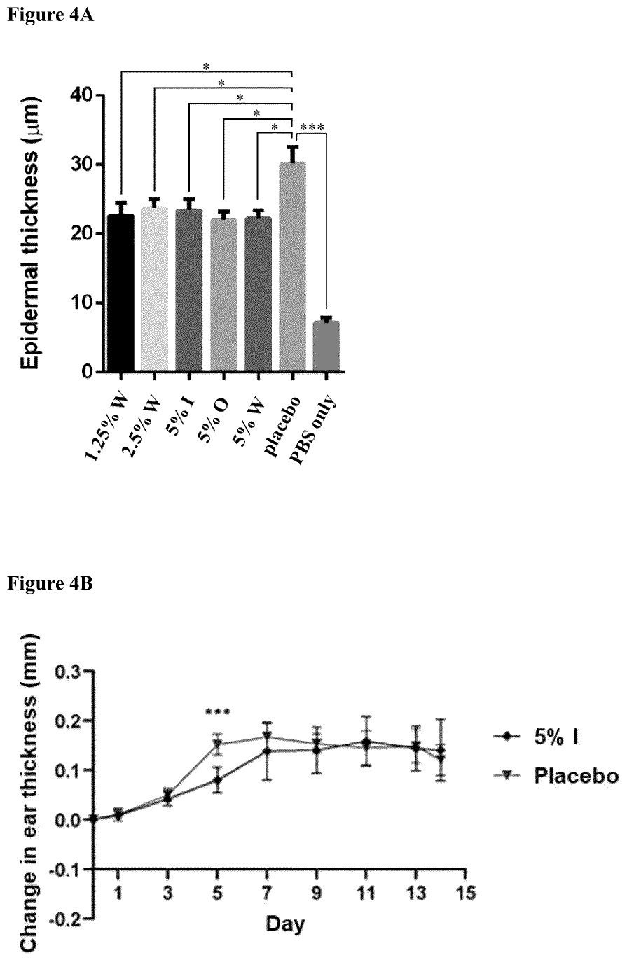

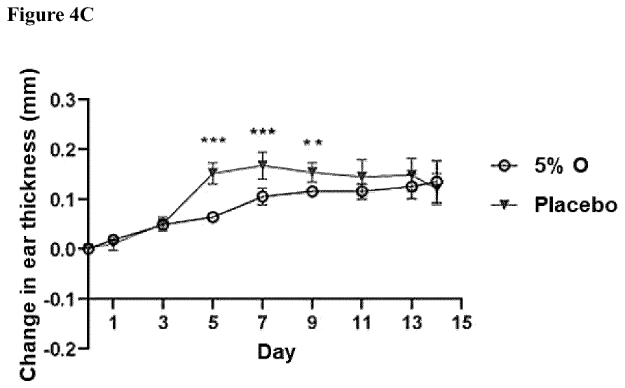

As shown in FIG. 4A, both inner shell extracts and outer shell extracts showed the potential effect to psoriasis. The results of the ear thickness of IL-23 injected right ear with 5% inner shell extracts and 5% outer shell extracts also showed significantly reduced ear swelling on day 5 (FIGS. 4B & 4C). In particular, administering with 5% outer shell extracts showed the reduction of the ear thickness for lasting longer time than administering with 5% inner shell extracts.

All the results demonstrated that treatment of these ointment samples were able to reduce the symptoms caused by psoriasis and one of them, 5% whole shell extracts, could reduce the ear thickness for lasting longer time.

While the invention has been described and exemplified in sufficient detail for those skilled in this art to make and use it, various alternatives, modifications, and improvements should be apparent without departing from the spirit and scope of the invention.

One skilled in the art readily appreciates that the present invention is well adapted to carry out the objects and obtain the ends and advantages mentioned, as well as those inherent therein. The cells, animals, and processes and methods for producing them are representative of preferred embodiments, are exemplary, and are not intended as limitations on the scope of the invention. Modifications therein and other uses will occur to those skilled in the art. These modifications are encompassed within the spirit of the invention and are defined by the scope of the claims.

1. A method for treating psoriasis, comprising administering a pharmaceutical composition comprises an effective amount of mangosteen fruit shell extract to a subject suffering from psoriasis.

2. The method of claim 1, wherein the extract of mangosteen fruit shell is water extract of mangosteen fruit shell and/or alcohol extract of mangosteen fruit shell.

3. The method of claim 1, wherein the mangosteen fruit shell is outer shell of the mangosteen fruit shell and/or inner shell of the mangosteen fruit shell.

4. The method of claim 1, wherein the mangosteen fruit shell is outer shell of the mangosteen fruit shell.

5. The method of claim 1, wherein the extract of mangosteen fruit shell comprises α-mangostin and γ-mangostin.

6. The method of claim 1, wherein the composition is a parenteral preparation.

7. The method of claim 6, wherein the parenteral preparation is an external preparation.

8. The method of claim 1, wherein the effective amount of extract of Mangosteen fruit shell is 1% w/w to 10% w/w.

9. The method of claim 1, wherein the effective amount of extract of Mangosteen fruit shell is 1% w/w to 8% w/w.

10. The method of claim 1, wherein the effective amount of extract of Mangosteen fruit shell is 1% w/w to 5% w/w.

11. The method of claim 1, wherein the psoriasis including plaque psoriasis, nail psoriasis, guttate psoriasis, inverse psoriasis, pustular psoriasis, erythrodermic psoriasis and psoriatic arthritis.

12. (canceled)

13. (canceled)
